(19) United States
(12) Patent Application Publication
(10) Pub. No.: US 2016/0038353 A1
(43) Pub. Date: Feb. 11, 2016

(54) SELECTION GUIDE AND ARRAY OF ABSORBENT ARTICLES

(71) Applicant: The Procter & Gamble Company, Cincinnati, OH (US)

(72) Inventors: Alejandro Javier BONILLA, Cincinnati, OH (US); Brice Daniel WESTRING, Mason, OH (US)

(21) Appl. No.: 14/741,500

(22) Filed: Jun. 17, 2015

Related U.S. Application Data
(60) Provisional application No. 62/034,464, filed on Aug. 7, 2014.

(51) Int. Cl. A61F 13/551; G09B 19/00; A61F 13/84

(52) U.S. Cl. CPC A61F 13/5514; A61F 13/55145; A61F 13/84; G09B 19/0076; A61F 2013/8497

(57) ABSTRACT

Selection guides for choosing an appropriate absorbent article are provided. The selection guide can include a reference line that communicates proper placement of the article, particularly where the article is asymmetrical. Arrays and packages of absorbent articles are also provided.

### FIELD OF THE INVENTION

The present invention relates to article selection guides, packages of absorbent articles comprising an article selection guide, and arrays of absorbent articles.

### BACKGROUND OF THE INVENTION

Consumers are often confused on what feminine hygiene article should be purchased to meet their needs. Store shelves include a plethora of products made by a variety of manufacturers. And each of the manufacturers offers several different sized and configured products, with naming and color schemes that are not always consistent from one manufacturer to another. And due to this inconsistency, consumers must spend an inordinate amount of time reading and studying information associated with multiple products to try to make the correct purchase decision.

Some manufacturers offer an overnight product. Many of these products are designed to be worn so that more of the product is positioned to the rear due to the direction bodily fluid flows when a consumer is lying down. But consumers may not understand this design intent and therefore may not adhere the product to their undergarment in a manner that takes advantage of this design intent. Some consumers just see the overnight product as a long product for greater protection needs. If consumers perceive an overnight product provides the highest level of protection simply due to its length and then wear the same during the daytime while not lying down, then a properly placed overnight product (more towards the rear) may not provide enough front protection. This misperception is not without basis because commercially available feminine hygiene articles do not carry a daytime designation to distinguish from overnight designated products.

Accordingly, there is much confusion of which feminine hygiene products are designed for daytime (when a consumer is an upright position) versus overnight (when a consumer is lying down), where to position feminine hygiene products, and which products are appropriate for a targeted level of protection. Embodiments of the present invention address at least some of this confusion.

### SUMMARY OF THE INVENTION

The present invention relates to absorbent article selection guides. In accordance one embodiment, there has now been provided an article selection guide, comprising a substrate or digital image; a reference line associated with the substrate or digital image; a first absorbent article image arranged on the reference line; and a second absorbent article image arranged on the reference line. The first absorbent article image is centered on the reference line. And the second absorbent article is offset with respect to the reference line so that more of the second absorbent article image resides on one side of the reference line versus an opposite side of the reference line. The article selection guides can be applied to a tangible substrate, such as, for example, a packaging material, and they can be presented to consumers in a digital format.

The present invention also relates to arrays of absorbent articles and arrays of packaged absorbent articles. In accordance with one embodiment, there has now been provided any array comprising a first package comprising a first absorbent article, a first brand name, a first color, and a designation that the first absorbent article is a daytime article; and a second package comprising a second absorbent article, a second brand name, a second color, and a designation that the second absorbent article is a nighttime article. The first absorbent article has a symmetrical shape, while the second absorbent article has an asymmetrical shape. The first brand name and the second brand name are the same. And the second color is a darker shade than the first color.

In accordance with a second embodiment, there has now been provided an array comprising a first package comprising a first feminine hygiene article, a first brand name, an extra heavy absorbency level designation, and an ultrathin or maxi designation; and a second package comprising a second feminine hygiene article, a second brand name, an extra heavy absorbency level designation, and an ultrathin or maxi designation. The second feminine hygiene article is different from the first feminine hygiene article. And the first brand name and the second brand name are the same.

In accordance with another embodiment, there has now been provided an array comprising a first collection of absorbent articles including a first absorbent article; a second absorbent article having a different length than that of the first absorbent article; and a third absorbent article having a different length that that of the first absorbent article and the second absorbent article; and a second collection of absorbent articles including a fourth absorbent article; and a fifth absorbent article having a different length than that of the fourth absorbent article. All of the first absorbent article, the second absorbent article and the third absorbent article are symmetrically-shaped and contained in packages comprising a daytime wear designation. Both the fourth absorbent article and the fifth absorbent articles are asymmetrically-shaped and contained in packages comprising a night time wear designation. The first collection of absorbent articles and the second collection of absorbent articles are sold under the same brand name.

### BRIEF DESCRIPTION OF THE DRAWINGS

The following detailed description of specific embodiments of the present invention can be best understood when read in conjunction with the drawings enclosed herewith.

FIG. 1 is an exemplary absorbent article selection guide.

FIG. 2 is another exemplary absorbent article selection guide.

FIG. 3 is a plan view of an exemplary symmetrically-shaped absorbent article.

FIG. 4 is a plan view of another exemplary symmetrically-shaped absorbent article.

FIG. 5 is a plan view of an exemplary asymmetrically-shaped absorbent article.

FIG. 6 is a front view of an array of packaged absorbent articles.

The embodiments set forth in the drawings are illustrative in nature and not intended to be limiting of the invention defined by the claims. Moreover, individual features of the drawings and invention will be more fully apparent and understood in view of the detailed description.

### DETAILED DESCRIPTION OF THE INVENTION

The following text sets forth a broad description of numerous different embodiments of the present invention. The description is to be construed as exemplary only and does not describe every possible embodiment since describing every possible embodiment would be impractical, if not impossible. And it will be understood that any feature, characteristic, component, composition, ingredient, product, step or methodology described herein can be deleted, combined with or substituted for, in whole or part, any other feature, characteristic, component, composition, ingredient, product, step or methodology described herein. Numerous alternative embodiments could be implemented, using either current technology or technology developed after the filing date of this patent, which would still fall within the scope of the claims. All publications and patents cited herein are incorporated herein by reference.

It should also be understood that, unless a term is expressly defined in this specification using the sentence “As used herein, the term ‘______’ is hereby defined to mean . . . ” or a similar sentence, there is no intent to limit the meaning of that term, either expressly or by implication, beyond its plain or ordinary meaning, and such term should not be interpreted to be limited in scope based on any statement made in any section of this patent (other than the language of the claims). No term is intended to be essential to the present invention unless so stated. To the extent that any term recited in the claims at the end of this patent is referred to in this patent in a manner consistent with a single meaning, that is done for sake of clarity only so as to not confuse the reader, and it is not intended that such a claim term be limited, by implication or otherwise, to that single meaning. Finally, unless a claim element is defined by reciting the word “means” and a function without the recital of any structure, it is not intended that the scope of any claim element be interpreted based on the application of 35 U.S.C. §112, sixth paragraph. As used herein, the term “absorbent article” refers to devices that can capture, absorb and/or contain liquid, and more specifically, refers to devices that are placed against or in proximity to the body of the wearer to absorb and contain the various exudates discharged from the body. A non-limiting list of absorbent articles includes feminine hygiene articles, diapers, incontinence products, and pants/briefs.

As used herein, the term “feminine hygiene article” refers to disposable absorbent articles to be worn by women for menstrual and/or incontinence control. Feminine hygiene articles are typically held in place adjacent the user's externally-visible genitalia (i.e., the pudendal region) by the user's undergarment. Feminine hygiene articles can be placed into the user's undergarment and affixed via adhesive or other joining means.

As used herein, the phrase “wear time” relates to the time of day an article is intended to be worn (daytime or nighttime)

As used herein, the phrase “protection level” relates to a targeted duration of protection the absorbent article can provide (e.g., up to 6 hours of protection, up to 8 hours of protection, up to 10 hours of protection).

The color scale values utilized herein to define the darkness/lightness of the materials of the absorbent article selection guides, packages, and absorbent articles are the widely accepted CIE LAB scale. Measurements can be made with a Hunter Color reflectance meter. A complete technical description of the system can be found in an article by R. S. Hunter, ‘photoelectric color difference Meter’, Journal of the Optical Society of America, Vol. 48, pp. 985-95, 1958. Devices specially designed for the measurement of color on the Hunter scales are described in U.S. Pat. No. 3,003,388 to Hunter et al., issued Oct. 10, 1961. When a color is defined according to this system L* represents lightness (0=black, 100=white), a* and b* independently each represent a two color axis, a* representing the axis red/green (+a=red, −a=green), while b* represents the axis yellow/blue (+b=yellow, −b=blue).

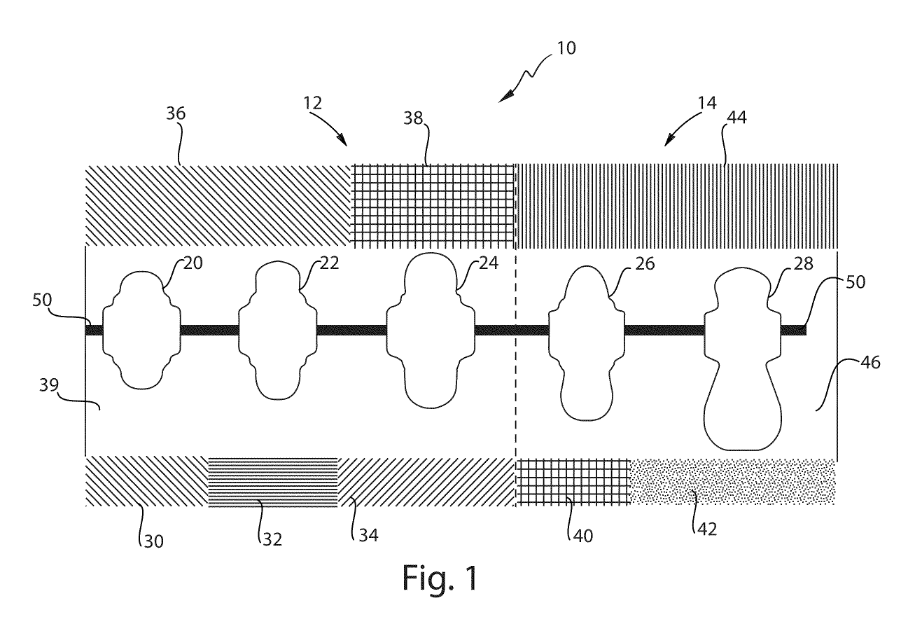

The present invention relates to absorbent article selection guides, packages of absorbent articles comprising a selection guide, arrays of absorbent articles, and arrays of packaged absorbent articles. Referring now to the figures, FIG. 1 shows an exemplary absorbent article selection guide 10 comprising a five article line-up including images of article 20, article 22, article 24, article 26, and article 28. The selection guide 10 can be provided in tangible form or digitally (e.g., via a website or digital advertisement). Tangible forms can include, but are not limited to, packaging, in-store features such as displays and shelf signage, store flyers, coupons, mailers, etc. The left hand region 12 of selection guide 10 includes articles 20, 22, and 24 that are designed for daytime usage. And the right hand region 14 includes articles 26 and 28 that are designed for nighttime usage.

Region 12 includes a number of different features to help consumers determine the right product for their needs. The lower portion of region 12 includes an absorbency level designation 30, 32, and 34 associated with a respective article shown in region 12. The absorbency level designation can comprise, for example, colors, words, phrases, symbols, other indicia and combinations thereof. By way of example only, absorbency level designation 30 includes a yellow color and the word “regular”, absorbency level designation 32 includes a green color and the words “long super”, and absorbency level designation 34 includes a coral/tangerine color and the words “extra heavy day”. The upper portion of region 12 includes a protection level and/or wear time designation 36 and 38. Protection level and/or wear time designation 36 applies to articles 20 and 22, while protection level and/or wear time designation 38 applies to article 24. Similar to the absorbency level designation, the protection level and wear time designations can include colors, words, phrases, symbols, other indicia, and combinations thereof. By way of example only, protection level and/or wear time designation 36 includes a yellow or orange color (to communicate the article is designed for daytime use), a sun symbol (to communicate the article is designed for daytime use), and an “up to 8 hour protection” designation (to communicate a protection level provided by the absorbent article); and protection level and/or wear time designation 38 includes a yellow or orange color (to communicate the article is designed for daytime use) and an “up to 10-hour protection” designation (to communicate a protection level provided by the absorbent article).

The lower portion of region 14 includes absorbency level designation 40 for article 26 and absorbency level designation 42 for article 28. In an exemplary embodiment, absorbency level designation 40 includes an orange color and the word “overnight”, and absorbency level designation 42 includes a purple color and the word “extra heavy overnight”. The upper portion of region 14 includes a protection level and/or wear time designation 44. Protection level and/or wear time designation can, for example, include a blue or black color (to communicate the article is designed for nighttime use), a moon symbol (to communicate the article is designed for nighttime use), star symbols (to communicate the article is designed for nighttime use), an “up to 10 hour protection” designation (to communicate a protection level provided by the absorbent article), or combinations of the same.

Regions 12 and 14 can also include a background color that communicates daytime and nighttime absorbent articles. For example, region 12 can have a color (e.g., green or blue) background 39 and region 14 can have a relatively darker color (e.g., a different shade or hue of green or blue) background 46. These colors and at least some of the above-mentioned designations are utilized on individual absorbent articles and/or wrappers or packages containing the absorbent articles to ensure a consumer purchases the article selected from the selection guide.

Absorbent article selection guide 10 also includes an optional reference line 50 that can represent an undergarment crotch central region. Articles 20, 22, 24, 26, and 28 are arranged on reference line 50 to communicate the proper placement of the designated article in one's undergarment. Articles 20, 22, and 24 are arranged so that about half of the pad length is on one side of reference line 50 and the other half of the pad length is on the opposite side of reference line 50. Articles 26 and 28 are arranged on reference line 50 so that more of the pad length is below reference line 50 and less of the pad length is above reference line 50 to communicate a more rearward positioning of articles 26 and 28 in one's undergarment.

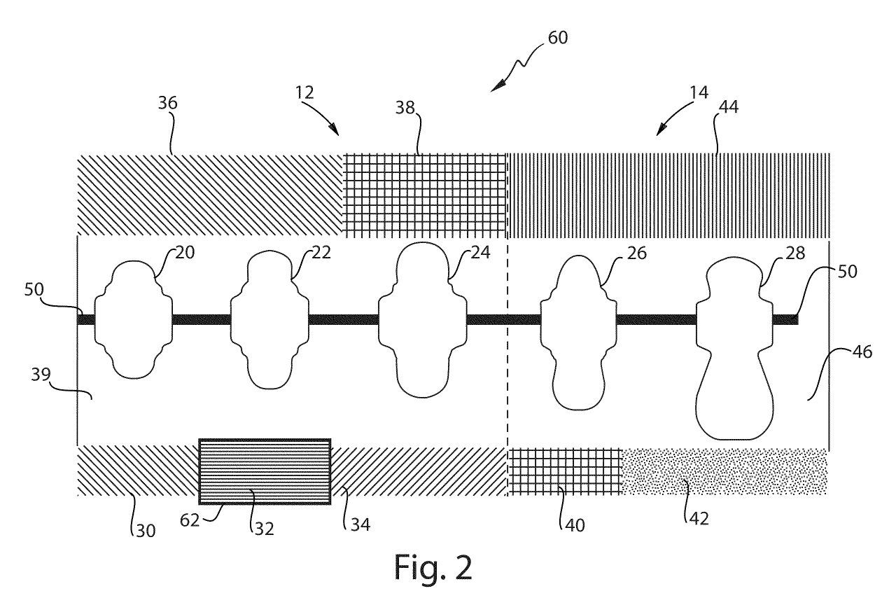

As noted previously, absorbent article selection guides of the present invention can be disposed on packages of absorbent articles. In this context, the article selection guide will optionally highlight the article(s) in the article line-up that is contained within the package. FIG. 2 shows an exemplary selection guide 60 that includes a frame 62 around absorbency level designation 32. One of skill in the art should appreciate that other techniques and approaches for highlighting the contained article can be employed. For instance, the article image itself or a geometric shape surrounding the article image could be highlighted.

The present invention also relates to absorbent article and package arrays corresponding to the article selection guides discussed above and variations thereof. As can be seen in FIG. 1, articles 20, 22, and 24 generally comprise a periphery having a symmetrical shape. And the side flaps or wings generally are centered on an article transverse centerline. Symmetry and asymmetry, as those terms are used herein, relate to the article shape on opposite sides of its transverse centerline and/or to the position of the side flaps or wings with respect to the article transverse centerline. An article is considered symmetrical or having a symmetrical shape when its side flaps or wings are less than 18, 15, 10, or 5 millimeters off center (side flap or wing transverse centerline in relation to the article transverse centerline).

Tables 1 and 2 below include a listing of exemplary ultrathin feminine hygiene articles and maxi feminine hygiene articles, respectively. Arrays of the present invention can include any combination of two or more of the articles either within the individual tables or across the individual tables. One of ordinary skill in the art should appreciate that absorbent articles other than those listed are contemplated by the present invention, including, for example, articles with dimensional variations in the range of plus or minus 10, 15, 20 millimeters.

TABLE 1
Ultrathin Feminine Hygiene Articles

| | | | Extra Heavy | | Extra Heavy |
|---|---|---|---|---|---|
| | Regular | Long Super | Day | Overnight | Overnight |
| Pad length (mm) | 240 | 284 | 317 | 317 | 360 |
| Pad length-front | 110 | 126 | 158.5 | 126 | 135 |
| from wing transverse | | | | | |
| centerline (mm) | | | | | |
| Pad length- back | 130 | 158 | 158.5 | 191 | 225 |
| from wing transverse | | | | | |
| centerline (mm) | | | | | |
| Overall pad width, | 158 | 158 | 180 | 158 | 158 |
| including wings | | | | | |
| (mm) | | | | | |
| Front lobe maximum | 102 | 104 | 101 | 104 | 101.8 |
| width (mm) | | | | | |
| Back lobe maximum | 102 | 104 | 101 | 104 | 120 |
| width (mm) | | | | | |
| Symmetric (S) or | S | S | S | A | A |
| Asymmetric (A) | | | | | |

TABLE 2
Maxi Feminine Hygiene Articles

| | | | Extra Heavy | | Extra Heavy |
|---|---|---|---|---|---|
| | Regular | Long Super | Day | Overnight | Overnight |
| Pad length (mm) | 240 | 284 | 317 | 317 | 400 |
| Pad length-front | 120 | 142 | 158.5 | 139.5 | 145 |
| from wing transverse | | | | | |
| centerline (mm) | | | | | |
| Pad length- back | 120 | 142 | 158.5 | 177.5 | 255 |
| from wing transverse | | | | | |
| centerline (mm) | | | | | |
| Overall pad width, | 160 | 160 | 180 | 160 | 170 |
| including wings | | | | | |
| (mm) | | | | | |
| Front lobe maximum | 90 | 90 | 107 | 88.5 | 125 |
| width (mm) | | | | | |
| Back lobe maximum | 90 | 90 | 107 | 108 | 175 |
| width (mm) | | | | | |
| Symmetric (S) or | S | S | S | A | A |
| Asymmetric (A) | | | | | |

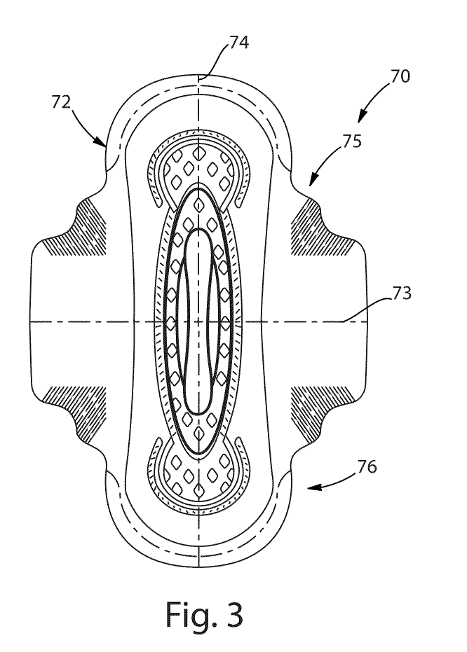

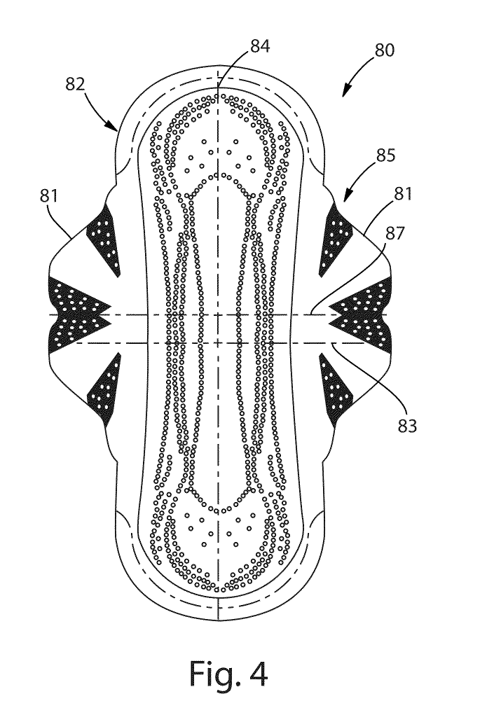

Absorbent articles of the present invention that have a daytime designation generally have a symmetrical shape to ensure there will be sufficient pad length in a frontward positioning when positioned properly within one's undergarment. FIGS. 3 and 4 illustrate exemplary symmetrically-shaped articles. FIG. 3 shows an exemplary symmetrical-shaped article 70 having a periphery 72, a transverse centerline 73, a longitudinal centerline 74, a first (e.g., front) portion 75 on one side of the transverse centerline, and a second (e.g., rear) portion 76 on the opposite side of the transverse centerline. As can be seen, the first portion 75 is substantially identical to the second portion 76 in both shape and size. FIG. 4 shows another exemplary symmetrical-shaped article 80 that includes side flaps or wings 81, a periphery 82, a transverse centerline 83, a longitudinal centerline 84, a first portion 85 on one side of the transverse centerline, and a second portion 86 on the opposite side of the transverse centerline. And while the periphery is designed such that the first portion 85 is substantially identical to the second portion 86 in both shape and size, a transverse centerline 87 of the side flaps 81 is slightly offset from the article transverse centerline 83. Articles of the present invention are still considered symmetrical wherein this offset is less than 15, 10, or 5 millimeters.

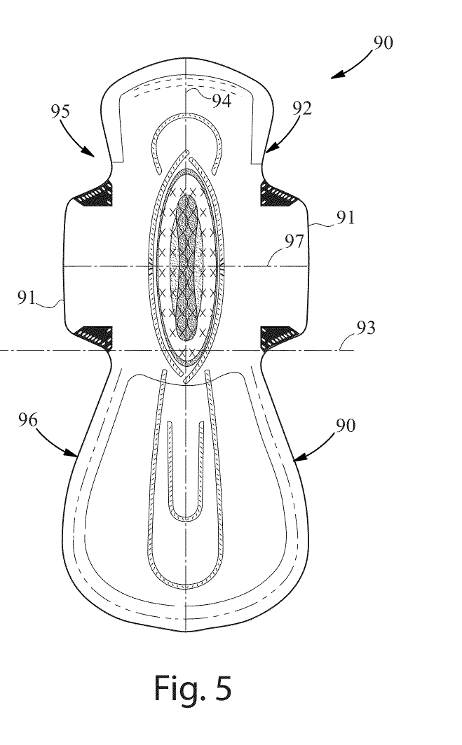

Referring again to FIG. 1, articles 26 and 28 are asymmetrical. These articles have a nighttime designation and have more surface area in their back portion and can be positioned more rearward to help capture bodily fluid when lying down—reference line 50 helps consumers visualize proper placement in their undergarment. FIG. 5 illustrates an exemplary asymmetrical-shaped article 90. Absorbent article 90 has a pair of side flaps 91, a periphery 92, a transverse centerline 93, a longitudinal centerline 94, a first portion 95 on one side of the transverse centerline, and a second portion 96 on the opposite side of the transverse centerline. The asymmetry of article 90 is two-fold: 1) the first portion 95 is significantly different in size and shaped from the second portion 96; and 2) the side flaps comprise a centerline 97 that is offset form the article transverse centerline 93 by 15 millimeters or more.

A variety of asymmetries are contemplated by the present invention, including along only one or both of the transverse centerline and the longitudinal centerline. The absorbent article in some embodiments is asymmetric about transverse centerline while being symmetric about longitudinal centerline, meaning that at least a portion of the first half and the second half are visibly configured differently (e.g., different size, shape, etc.) and/or are configured differently in a manner that is not readily discernible by simply looking at the article (e.g., different exudates management properties or physical properties).

Absorbent articles of the present invention typically include a topsheet, a backsheet, and an absorbent core disposed therebetween. The topsheet can be formed from any generally soft, compliant, and porous material which is comfortable against human skin and through which bodily exudates can pass. Exemplary topsheets include nonwovens and apertured polymeric films. Backsheets are generally configured to hinder or prevent bodily exudates from penetrating all the way through the absorbent article and soiling a user's garments or body. The backsheet can be liquid impermeable and be formed from polymer films or film/nonwoven laminates. Backsheets can be vapor permeable while being liquid impermeable to help render the article more breathable. Exemplary vapor permeable materials include microporous films, apertured formed films, and nonwovens. Such materials may include an additive (e.g., wax or surfactant) or treatment to increase their hydrophobicity or hydrophilicity. The absorbent core can be formed from any of the materials well known to those of ordinary skill in the art. Examples of such materials include, but are not limited to, plies of creped cellulose wadding, fluffed cellulose fibers, wood pulp fibers known as airfelt, a mass or batt of fibers, airlaid webs or fibers, a web of polymeric fibers, and a blend of polymeric fibers. The absorbent core can also comprise high capacity materials such as polyacrylate particles (often referred to as “superabsorbents” and absorbent gelling material (AGM)) and HIPE foams like those disclosed in U.S. Pat. Nos. 5,550,167; 5,387,207; 5,352,711; and 5,331,015.

One or more optional intermediate layers can be employed in the absorbent articles of the present invention. In one configuration, an optional intermediate layer can be disposed between the topsheet and the absorbent core. Such layers are included in many known absorbent article designs and can be referred to as “secondary topsheets,” “surge layers,” “acquisition layers,” “distribution layers,” “transport layers,” “wicking layers” and the like. With this placement, the optional intermediate layer serves to help rapid acquisition of bodily exudates from the topsheet and movement of the same into the absorbent core, and/or to temporarily hold bodily exudates as a means for managing large loading volumes or fast loading rates. Exemplary surge layers are disclosed in U.S. Pat. Nos. 5,843,063 and 5,879,343. In another configuration, an optional intermediate layer can be disposed between the absorbent core and the backsheet. In this location, an intermediate layer may provide any number of benefits including, for example, structural integrity, flexibility, body shaping, and bodily exudates management. Absorbent articles of the present invention can be individually wrapped. The wrapping material can help provide cleanliness and discretion when they are carried outside of the home. The wrappers generally at least partially enclose the absorbent article, and preferably, substantially completely enclose the article. In some embodiments, the wrapper is configured to accept a used article to facilitate disposal of the same. The wrappers can be constructed from numerous materials, including, for example, polymeric films, fibrous materials (including nonwovens and wovens), paper, card stock, and combinations thereof. In one preferred embodiment, the wrappers are constructed from flexible, polymeric films. The polymeric films may be based on polyethylene, polypropylene, polyester, nylon, polyvinyl alcohol, or blends of the same. One exemplary material is a 32 gauge polypropylene film. The materials may be a single layer or more than one layer. The wrappers can be formed from a single feedstock of material that is manipulated into a container, such as by folding, or can be formed from multiple feedstocks that are joined together to ultimately form the finished wrapper.

The wrappers can include one or more colors and/or other indicia that communicate valuable purchase and use decisions of the absorbent articles. And the colors/indicia of daytime articles can be related to the colors/indicia of nighttime articles. For example, an extra heavy day feminine hygiene article can be individually wrapped in a coral/tangerine shade having an L* value of from about 58 to about 63, an a* value of from about 55 to about 68, and a b* value of from about 54 to about 65, while an overnight feminine hygiene article can be individually wrapped in an orange shade having an L* value of from about 73 to about 84, an a* value of from about 9 to about 21, and a b* value of from about 67 to about 79. These distinct but closely coordinated wrapper colors help consumers understand one is a particular coverage level designed for daytime use and the other is a similar coverage level but designed for nighttime use. One way of coordinating wrapper colors for two or more articles is to utilize color schemes that consistently use a positive or negative a* value and/or a positive or negative b* value; for example, a first wrapper having a color exhibiting a positive a* value and a second wrapper having a color exhibiting a positive a* value. Delta E* values can be generated from the above L*, a*, b* values to provide more color shade coordination options contemplated by the present invention.

Packages of the present invention contain a plurality of individually wrapped articles. Materials for constructing the packages and the package configurations can be any of those known by the skilled artisan. Examples include, but are not limited to, paper or board stock cartons and polymeric (e.g., polypropylene) film bags. The packages can optionally contain a window that may allow at least some of the wrapper printed elements to be seen therethrough. “Window(s)”, as that term is used herein, can include complete through openings, transparent sections, translucent sections, film inserts/coverings in a cardboard carton cutout, and the like. In some embodiments, the packages contain a plurality of the same article. And other embodiments, there packages can contain a plurality of different articles.

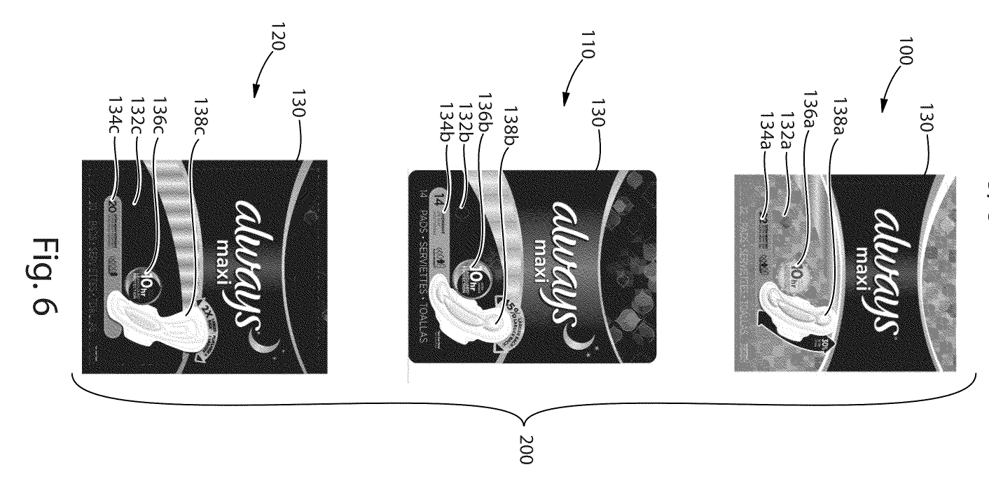

FIG. 6 shows an exemplary array 200 of three packages 100, 110, and 120 of absorbent articles. Each of the packages comprises a brand name region 130 comprising the ALWAYS® brand name, a background color 132 (132a on package 100, 132b on package 110, and 132c on package 120), an absorbency level designation 134 (134a on package 100, 134b on package 110, and 134c on package 120), a first icon 136 (136a on package 100, 136b on package 110, and 136c on package 120), and a second icon 138 (138a on package 100, 138b on package 110, and 138c on package 120).

Package 100 contains absorbent articles designed for daytime usage, while packages 110 and 120 contain absorbent articles designed for nighttime usage. The background color 132b and 132c is a darker shade or hue than the background color 132a. In one exemplary embodiment, the lighter background shade can be a green shade having an L* value of from about 51 to about 65, an a* value of from about −43 to about −40, and a b* value of from about 3 to about 9, while the darker background shade is a green shade having an L* value of from about 11 to about 42, an a* value of from about −33 to about −17, and a b* value of from about 6 to about 13. In a second exemplary embodiment, the lighter background shade is a blue shade having an L* value of from about 43 to about 68, an a* value of from about −29 to about −7, and a b* value of from about −41 to about −30, while the darker background shade is a blue shade having an L* value of from about 10 to about 31, an a* value of from about −7 to about 1, and a b* value of from about −31 to about −24. Delta E* values can be generated from the above L*, a*, b* values to provide more color shade differences contemplated by the present invention.

The absorbency level designation 134a on package 100 comprises “extra heavy day” and 134c on package 120 comprises “extra heavy overnight”. And the absorbency level designation 134b on package 110 comprises “overnight”. The first icon 136a on package 100 comprises a sun image and an “up to 10 hour protection” designation, while the first icon 136b and 136c comprise a moon image and an “up to 10 hour protection” designation to help the consumer make informed purchase decisions. The second icon 138 includes an image of the contained absorbent article.

One of ordinary skill in the art should appreciate that while much of the above discussion has focused on feminine hygiene articles, other types of absorbent articles are contemplated by the present invention, and the article/package arrays of the present invention can include different types of articles.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application and any patent application or patent to which this application claims priority or benefit thereof, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A package of absorbent articles, the package comprising:
a. package material configured to define an interior;
b. a plurality of absorbent articles disposed in the interior; and
c. an article selection guide disposed on the package material, the article selection guide comprising:
i. a reference line;
ii. a first absorbent article image arranged on the reference line, the first absorbent article image being substantially centered on the reference line; and
iii. a second absorbent article image arranged on the reference line, the second absorbent article image being offset with respect to the reference line so that more of the second absorbent article image resides on one side of the reference line versus an opposite side of the reference line.

2. The package of claim 1, wherein one of the first absorbent article image and the second absorbent article image is highlighted to indicate what type of absorbent article is contained within the package.

3. An article selection guide, comprising:
a. a substrate or digital image;
b. a reference line associated with the substrate or digital image;
c. a first absorbent article image arranged on the reference line, the first absorbent article image being substantially centered on the reference line; and
d. a second absorbent article image arranged on the reference line, the second absorbent article image being offset with respect to the reference line so that more of the second absorbent article image resides on one side of the reference line versus an opposite side of the reference line.

4. The selection guide of claim 3, wherein the reference line represents an undergarment crotch central region.

5. The selection guide of claim 3, further comprising a daytime designation in proximity to the first absorbent article image.

6. The selection guide of claim 3, further comprising a nighttime designation in proximity to the second absorbent article image.

7. The selection guide of claim 3, wherein a protection level designation is associated with at least one of the first absorbent article image and the second absorbent article image.

8. The selection guide of claim 3, wherein an absorbency level designation is associated with each of the first absorbent article image and the second absorbent article image.

9. The selection guide of claim 8, wherein the absorbency level designation associated with the first absorbent article image is the same as the absorbency level designation associated with the second absorbent article image.

10. An array of packaged absorbent articles, the array comprising:
a. a first package comprising a first absorbent article, a first brand name, a first color, and a designation that the first absorbent article is a daytime article; and
b. a second package comprising a second absorbent article, a second brand name, a second color, and a designation that the second absorbent article is a nighttime article;
c. wherein the first absorbent article has a symmetrical shape;
d. wherein the second absorbent article has an asymmetrical shape;
e. wherein the first brand name and the second brand name are the same; and
f. wherein the second color is a darker shade than the first color.

11. The array of claim 10, wherein the first package and the second package comprise the same absorbency level designation.

12. The array of claim 11, wherein the absorbency level designation comprises “extra heavy”.

13. The array of claim 10, wherein the first color and second color are blue.

14. The array of claim 10, wherein the first color and the second color are green.

15. The array of claim 10, wherein the first package comprises a first protection level designation and the second package comprises a second protection level designation.

16. The array of claim 15, wherein the first protection level designation is the same as the second protection level designation.

17. An array of packaged absorbent articles, the array comprising:
a. a first package comprising a first feminine hygiene article, a first brand name, an “extra heavy” absorbency level designation, and an ultrathin or maxi designation; and
b. a second package comprising a second feminine hygiene article, a second brand name, an “extra heavy” absorbency level designation, and an ultrathin or maxi designation;
c. wherein the second feminine hygiene article is different from the first feminine hygiene article; and
d. wherein the first brand name and the second brand name are the same.

18. The array of claim 17, wherein the first feminine hygiene article has a symmetrical shape and the second feminine hygiene article has an asymmetrical shape.

19. The array of claim 18, wherein the first feminine hygiene article has a length that is substantially the same as that of the second feminine hygiene article.

20. The array of claim 17, wherein the first feminine hygiene article is wrapped in a first wrapper having a first color and the second feminine hygiene article is wrapped in a second wrapper having a second color, and wherein the first and second color have CIE a* value differences of less than 30 and/or CIE b* value differences of less than 20.

21. The array of claim 17, wherein the first feminine hygiene article is an ultrathin feminine hygiene article and the second feminine hygiene article is an ultrathin feminine hygiene article.

22. The array of claim 17, wherein the first feminine hygiene article is a maxi feminine hygiene article and the second feminine hygiene article is a maxi feminine hygiene article.

23. An array of absorbent articles, comprising:
a. a first collection of absorbent articles comprising:
i. a first absorbent article;
ii. a second absorbent article having a different length than that of the first absorbent article; and
iii. a third absorbent article having a different length that that of the first absorbent article and the second absorbent article;
iv. wherein all of the first absorbent article, the second absorbent article and the third absorbent article are symmetrically-shaped and contained in packages comprising a daytime wear designation;
b. a second collection of absorbent articles comprising:
i. a fourth absorbent article; and
ii. a fifth absorbent article having a different length than that of the fourth absorbent article;
iii. wherein both the fourth absorbent article and the fifth absorbent articles are asymmetrically-shaped and contained in packages comprising a night time wear designation;
c. wherein the first collection of absorbent articles and the second collection of absorbent articles are sold under the same brand name.

24. The array of claim 23, wherein packages comprising the first absorbent article and the second absorbent article have a protection level designation of up to 8 hours and the package of the third absorbent article has a protection level designation of up to 10 hours.

25. The array of claim 23, wherein the third absorbent article has the same length as the fourth absorbent article.
